(19) United States
(12) Patent Application Publication
(10) Pub. No.: US 2019/0172571 A1
(43) Pub. Date: Jun. 6, 2019

(54) ENHANCED ASSISTIVE MOBILITY DEVICES

(71) Applicant: Elements of Genius, Inc., Mt. Pleasant, SC (US)

(72) Inventor: Jonathan E. Ramaci, Mt. Pleasant, SC (US)

(21) Appl. No.: 16/206,475

(22) Filed: Nov. 30, 2018

Related U.S. Application Data
(60) Provisional application No. 62/593,305, filed on Dec. 1, 2017.

(51) Int. Cl. G16H 10/60; G16H 20/30; G08B 21/04

(52) U.S. Cl. CPC G16H 10/60; G16H 20/30; G08B 21/043; G08B 21/0461; A61G 2203/20; A61H 2201/5084; A61H 2201/5043; A61H 2201/0157; A61H 2201/501

(57) ABSTRACT

A system and apparatus for a mobile communication device that is attachable to one or more assistive mobility device. The device provides a user-interface that allows a user to access features that include smart and secure location-based services, mobile phone module, voice and data, advanced battery system and power management, direct 911 access, and fall detection via an accelerometer sensor. Additional functions may include one or more measurements of linear acceleration, heading, altitude, angular velocity, and angular position. The wearable device may contain one or more microprocessor, microcontroller, micro GSM/GPRS chipset, micro SIM module, read-only memory device (ROM), RAM, memory storage device, I-O devices, buttons, display, user interface, rechargeable battery, microphone, speaker, audio CODEC, power gauge monitor, wireless battery charger, wireless transceiver, antenna, accelerometer, vibrating motor, LEDs, preferably in combination, to function fully as a wearable mobile cellular phone.

### CROSS REFERENCE TO RELATED APPLICATIONS

This application claims the benefit of U.S. Provisional Application 62/593,305, entitled “ENHANCED ASSISTIVE MOBILITY DEVICES,” filed Dec. 1, 2017 and hereby incorporated by reference in its entirety.

### FIELD

The present disclosure relates to the field of enhanced assistive mobility devices and methods of assisting users with a voice-controlled relational agent.

### BACKGROUND

Inactivity among the elderly can increase morbidity and premature mortality, and devices that enable daily active mobility are essential to their health and wellbeing. The ability to move independently represents a hallmark of autonomous living and quality of life (QoL), while being physically active is associated with positive health outcomes. However, sensory, motor or cognitive impairments restrict mobility in the frail elderly population. Assistive mobility devices (AMDs) are often prescribed for and used by older adults to enhance mobility, compensate for decrements in balance, coordination, sensation, strength, reduce the risk of fall, extend independent living, and improve QoL. These devices include canes, standard and wheeled walkers, manually propelled and motorized wheelchairs, and scooters.

Cognitive function plays a key role in the regulation and control of routine walking, especially in older adults. Attention is a necessary cognitive resource for maintaining normal walking and navigation, and attentional deficits are independently associated with postural instability and impairment in performing activities of daily living. Evidence indicates that impaired cognitive processes, particularly cognitive flexibility and working memory, are prevalent in older people and associated with falls. Mobility impairments can also restrict the capacity for social interactions, which is also an important factor associated with maintaining cognitive function.

Walking is traditionally viewed as an automatic motor task that requires minimal higher mental functions. It is now understood that normal walking requires strategic planning of the best route, as well as continuous interaction with the environment and with internal factors. The safety and efficacy of normal walking critically depend on the interaction between the executive control dimension e.g., (integration and decision of action) with the cognitive dimension (e.g., navigation, visuospatial perception, or attention) and the affective dimension (e.g., mood, cautiousness). Such an integration is challenged is when people must walk while performing one or more secondary tasks. In the elderly, this dual task ability deteriorates due to the decline in central thinking resources that is secondary to subclinical disease processes or medication.

Among the available AMDs, walkers have many users including the elderly because of its simplicity and rehabilitation potential. A walker typically consists of a rigid frame having a forward pair of legs, a rear pair of legs, and a handle means located at the upper portion by which the user grasps the walker. Other configurations are also known, such as walkers having one or more pairs of wheels to assist with movement of the walker. These devices can increase confidence and sense of safety, which can raise a user's level of activity and independence. There may be physiological benefits of limiting osteoporosis and improving peripheral circulation as well as psychological benefits of maintaining self-esteem and social relationship.

Walkers are differentiated in terms of technological complexity, size, and structure. Conventional and smart walkers represent most walkers with the conventional being passive, simple in structure, and low cost. Rollators are a class of walkers typified by a walking frame with either two, three, or four wheels. Despite the dependence on such ambulatory assisting devices, conventional walkers and variants only provide assistance with user stability. Navigational assistance is not an available feature in walkers for users suffering from senile dementia as well as possessing deficiencies in motor skills.

Smart walkers have emerged with similar architecture as conventional ones but including additional robotic and electronic components. Smart walkers have evolved to provide assistance to the user at different levels, depending on the user's needs and with following functionalities: physical support, sensory assistance, cognitive assistance, health monitoring, and advanced human-machine interface. Smart walkers can also be classified according to their capability to assist user navigation and (auto-) localization in structured environments and outdoors (e.g., using GPS). Smart walkers may also be able to communicate bidirectionally with the user through a visual interface or voice commands, receiving directions from the user, or informing him about the present localization in a map and the environmental conditions including obstacles. However, these devices in general are complex to use and designed without consideration for elderly users possessing cognitive and sensory impairments as well as the challenge of using sophisticated mechanical-electrical devices.

Thus, there is a need to augment conventional AMDs with functions and capabilities to assist the user with activities of daily living and or rehabilitation beyond providing only physical and stability support. It is evident that mobility in the elderly may not only be restricted by motor capabilities but also by sensorial and or cognitive impairments. Successful device-assisted rehabilitation and management of mobility difficulties should target both physical functions and cognitive processes. In view of the complex interactions between walking, cognition, and mood, new interventional strategies may promote secured mobility of elderly people by improving attention, dual task performance, mood, and executive functions, as well as providing, among others, orientation/navigation guidance. These strategies should incorporate the use of a simple, user-friendly, natural, and low cognitive demand user-interface, especially for the elderly.

### SUMMARY

In an aspect of the present disclosure, the functions and capabilities of assistive mobility devices (AMDs) are augmented through an assistive technology platform (system). In the broadest terms, the platform incorporates at least one device attachable or detachable to or from an AMD, providing one or more user functions including, but not limited to, voice, data, SMS reminders, alerts, medication adherence monitoring, location via SMS, GPS location/navigation, fall detection, and 911 emergency services. The said device incorporates one or more microprocessor, microcontroller, micro GSM/GPRS chipset, micro SIM module, read-write memory device, read-only memory device (ROM), random access memory (RAM), memory storage device, I-O devices, buttons, display, user interface, rechargeable battery, microphone, speaker, wireless transceiver, RF electronic circuits, audio CODEC, cellular antenna, GPS antenna, accelerometer, vibrating motor(output), preferably in combination, to function fully as a wireless mobile cellular communication unit. The device can perform one or more executable codes, algorithms, methods, and or software instructions for automated voice recognition-response, natural language understanding-processing, and wireless mobile cellular communication.

According to another aspect of the present disclosure, the said device may function in combination with an application software platform accessible to multiple clients (users) executable on one or more remote servers, to preferably establish a communication ecosystem. The ecosystem enables communication and social networking for an AMD user, family, caregivers, and or healthcare providers. Furthermore, the device may function in combination with one or more remote servers, cloud control services, to perform natural language or speech-based interactions with the user, preferably through a voice-controlled speech user interface.

According to another aspect of the present disclosure, the voice-controlled speech user interface of said device detects or monitors audio input/output and interacts with a user to determine a user intent based on natural language understanding of the user's speech. The voice-controlled speech user interface is configured to capture user utterances and provide them to the control service. The combination of the speech interface device and one or more applications executed by the control service serves as a relational agent. The relational agent provides conversational interactions, utilizing automated voice recognition-response, natural language processing, predictive algorithms, and the like, to interact with the user and fulfill user requests, preferably providing cognitive aids for improving attention, dual task performance, mood, and executive functions, as well as providing, among others, orientation and navigation guidance, for safe and secured rehabilitation or mobility management.

According to yet another aspect of the present disclosure, the said device is attachable/detachable to one or more AMDs to augment their functions and capabilities, preferably through several following embodiments. In one embodiment, the AMD is a cane. In another embodiment, the AMD is a crutch. In yet another embodiment, the AMD is a walker. In an alternative embodiment, the AMD is a rollator. In yet another embodiment, the AMD is a scooter. The said augmented AMDs provide user access to one or more functions including, but not limited to, voice, data, SMS reminders, alerts, medication adherence monitoring, location via SMS, GPS location/navigation, navigation guidance, fall detection, and 911 emergency services. In addition, the augmented AMDs enable the user to access and interact with the said relational agent and communication ecosystem for safe and secured rehabilitation or mobility management.

According to another aspect of the present disclosure, the said device can communicate with a secured HIPAA-compliant remote server. The remote server is accessible through one or more computing devices, including but not limited to, desk-top, laptop, tablet, mobile phone, smart appliances (i.e. smart TVs), and the like. The remote server contains a well-being support application software that includes a database for storing user (s) information. The application software provides a collaborative working environment to enable a voluntary, active, and collaborative effort between an AMD user, health care team/providers, caregivers, and family members. The software environment allows for, but is not limited to, daily tracking of patient location, monitoring of medication adherence, sending-receiving text messages, push notifications, sending-receiving voice messages, sending-receiving videos, streaming instructional videos, scheduling doctor's appointments, patient education information, caregiver education information, feedback to healthcare providers, and the like. The application software can be used to store skills relating to the self-management of activities of daily living, rehabilitation, and or physical therapy. The application software may contain functions for predicting or monitoring AMD user/patient behaviors, gait, falls, non-compliance, non-compliance to pharmacologic therapy, non-compliance to physical therapy, functions for suggesting corrective actions, functions for performing or providing cognitive aids for improving attention, dual task performance, mood, executive functions, orientation, and navigational guidance with visual or auditory sensory cues. The application software may interact with an electronic health or medical record system (e.g., EMR).

According to another aspect of the present disclosure, the said secured remote server is also accessible using a stand-alone voice-controlled speech user interface device or a speech user interface incorporated into one or more smart appliances, or mobile apps, capable of communicating with the same or another remote server, providing cloud-based control service, to perform natural language or speech-based interaction with the user, acting as said relational agent. The relational agent provides conversational interactions, utilizing automated voice recognition-response and natural language learning-processing, to perform various functions and the like, to: interact with the user, fulfill user requests, educate, monitor compliance, monitor persistence, provide one or more skills, ask one or more questions, store responses/answers, perform predictive algorithms with user responses, determine health status and well-being, and provide suggestions for corrective actions including instructions to promote safe and secured rehabilitation or mobility management.

According to yet another aspect of the present disclosure, the said skills are developed and accessible through the said relational agent. These skills may include but are not limited to specific educational topics, nutrition (e.g., glycemic index, etc.), instructions for taking medication, improving medication adherence, gaiting, walking, physical rehabilitation, improving cognition, increasing persistence, symptoms management, proprietary developed skills, coping skills, behavioral skills, skills for daily activities, skills for caregivers, skills for improving attention, dual task performance, mood, executive functions, orientation, and navigational guidance, with other skills becoming apparent to one skilled in the art upon review of the present disclosure.

According to another aspect of the present disclosure, the AMD user interacts with the relational agent via providing responses or answers to clinically validated questionnaires or instruments. The questionnaires enable the monitoring of patient behaviors, physical rehabilitation compliance, physical recovery progress, cognitive functions, sensory functions, medication compliance, medication adherence, medication persistence, wellness, symptoms, adverse events monitoring, and the like. The responses or answers provided to the relational agent serve as input to one or more predictive algorithms to calculate a risk stratification profile and trends. Such a profile can provide an assessment for the need of any intervention or behavior modification required by either the user, caregivers, family members, or healthcare team/providers.

Specific embodiments of the present disclosure provide for an assistive mobility system comprising a portable user interface device operably engaged with a communications network, the portable user interface device configured to be selectively coupled to a surface of an assistive mobility device, the portable user interface device comprising at least one processor operably engaged with at least one non-transitory computer readable medium, a user interface comprising one or more input/output means operably engaged with the at least one processor, at least one accelerometer engaged with the at least one processor, at least one microphone and at least one speaker operably engaged with the at least one processor, the at least one non-transitory computer readable medium having instructions stored thereon to cause the processor to process one or more user inputs, and perform one or more assistive mobility actions in response to the one or more user inputs; and, a remote application server being communicably engaged with the portable user interface device via the communications network to receive a data transmission associated with one or more user inputs, the remote application server executing a control service comprising an automated speech recognition function, a natural-language processing function, and an application software, the application software executing one or more routines in response to the data transmission, the one or more routines comprising instructions for delivering one or more assistive mobility prompts to the portable user interface device.

Further specific embodiments of the present disclosure provide for an assistive mobility system comprising an assistive mobility device; a portable user interface device operably engaged with a communications network, the portable user interface device being selectively coupled to a surface of the assistive mobility device, the portable user interface device comprising at least one processor operably engaged with at least one non-transitory computer readable medium, a user interface comprising one or more input/output means operably engaged with the at least one processor, at least one accelerometer engaged with the at least one processor, at least one microphone and at least one speaker operably engaged with the at least one processor, the at least one non-transitory computer readable medium having instructions stored thereon to cause the processor to monitor a plurality of user activity data and execute one or more physical rehabilitation or mobility management interventions in response to the plurality of user activity data; and, a remote application server being communicably engaged with the portable user interface device via the communications network to receive the plurality of user activity data, the remote application server executing a control service comprising an automated speech recognition function, a natural-language processing function, and an application software, the application software executing one or more routines in response to the plurality of user activity data, the one or more routines comprising instructions for evaluating user compliance with one or more physical rehabilitation or mobility management parameters.

Still further specific embodiments of the present disclosure provide for an assistive mobility system comprising an assistive mobility device comprising an assistive mobility assembly and an electronics assembly being operably engaged with a communications network, the electronics assembly comprising at least one processor operably engaged with at least one non-transitory computer readable medium, a user interface comprising one or more input/output means operably engaged with the at least one processor, at least one accelerometer engaged with the at least one processor, at least one microphone and at least one speaker operably engaged with the at least one processor, the at least one non-transitory computer readable medium having instructions stored thereon to cause the processor to monitor a plurality of user activity data and execute one or more physical rehabilitation or mobility management interventions in response to the plurality of user activity data; and, a remote application server being communicably engaged with the assistive mobility device via the communications network to receive the plurality of user activity data, the remote application server executing a control service comprising an automated speech recognition function, a natural-language processing function, and an application software, the application software executing one or more routines in response to the plurality of user activity data, the one or more routines comprising instructions for evaluating user compliance with one or more physical rehabilitation or mobility management parameters.

In summary, the device and the assistive technology platform (system) of the present disclosure serve to augment the functions and capabilities of AMDs and provide a social, caregiver, healthcare provider(s) network support system for the AMD user. The system incorporates a voice-controlled empathetic relational agent as a simple, user-friendly, natural, and low cognitive demand user-interface. The device and system enable the enhanced assistance to the AMD user in the rehabilitation and or management or recovery of safe and secured mobility.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity, wherein:

FIG. 1 is a schematic diagram illustrating the components of the cellular communication device to augment the functions and capabilities of AMDs;

FIG. 2 is an illustration of a simple user interface in accordance with an aspect of the present disclosure;

FIG. 3 is an illustration depicting a conventional cane with the communication device attached to its frame;

FIG. 4 is an illustration depicting a conventional crutch with the communication device attached to its frame;

FIG. 5 is an illustration depicting a conventional walker with the communication device attached to its frame;

FIG. 6 is an illustration depicting a conventional rollator with the communication device attached to its frame;

FIG. 7 is an illustration depicting a conventional scooter with the communication device attached to its frame;

FIG. 8 is an illustration containing the elements of a communication ecosystem;

FIG. 9 is a screen-shot illustrating information that users can generate using the application software platform according to an aspect of the present disclosure;

FIG. 10 illustrates the assistive technology platform (system) incorporating a multimedia device; and,

FIG. 11 illustrates a figurative relational agent comprising the voice-controlled speech user interface of the cellular communication device.

### DETAILED DESCRIPTION

The present disclosure can be understood more readily by reference to the following detailed description, examples, drawings, and claims, and their previous and following description. However, before the present devices, systems, and/or methods are disclosed and described, it is to be understood that this disclosure is not limited to the specific devices, systems, and/or methods disclosed unless otherwise specified, as such can, of course, vary. It is also to be understood that the terminology used herein is for the purpose of describing particular aspects only and is not intended to be limiting.

The following description is provided as an enabling teaching of the present devices, systems, and/or methods in their best, currently known aspect. To this end, those skilled in the relevant art will recognize and appreciate that many changes can be made to the various aspects described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout.

The terminology used herein is for describing particular embodiments only and is not intended to be limiting. As used herein, the singular forms “a,” “an,” and “the” are intended to include the plural forms as well, unless the context clearly indicates otherwise. It will be further understood that the terms “comprises,” “comprising,” “includes,” and/or “including,”, and variants thereof, when used herein, specify the presence of stated features, steps, operations, elements, and/or components, but do not preclude the presence or addition of one or more other features, steps, operations, elements, components, and/or groups thereof.

It will be understood that when an element is referred to as being “coupled,” “connected,” or “responsive” to another element, it can be directly coupled, connected, or responsive to the other element, or intervening elements may also be present. In contrast, when an element is referred to as being “directly coupled,” “directly connected,” or “directly responsive” to another element, there are no intervening elements present. As used herein, the term “and/or” includes any and all combinations of one or more of the associated listed items.

Spatially relative terms, such as “above,” “below,” “upper,” “lower,” “top, “bottom,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” other elements or features would then be oriented “above” the other elements or features. Thus, the term “below” can encompass both an orientation of above and below. The device may be otherwise oriented (rotated 90 degrees or at other orientations) and the spatially relative descriptors used herein interpreted accordingly. Well-known functions or constructions may not be described in detail for brevity and/or clarity.

It will be understood that, although the terms “first,” “second,” etc. may be used herein to describe various elements, these elements should not be limited by these terms. These terms are only used to distinguish one element from another. Thus, a first element could be termed a second element without departing from the teachings of the present embodiments.

Unless otherwise defined, all terms (including technical and scientific terms) used herein have the same meaning as commonly understood by one of ordinary skill in the art to which these embodiments belong. It will be further understood that terms, such as those defined in commonly-used dictionaries, should be interpreted as having a meaning that is consistent with their meaning in the context of the relevant art and will not be interpreted in an idealized or overly-formal sense unless expressly so defined herein.

Numerous alternative embodiments of the device and technology platform (system) to augment AMDs are described herein. Such a device and system may help make users more independent and healthier, as well as facilitate the rehabilitation and or management or recovery of safe and secured mobility with greater ease and convenience. An aspect of the present disclosure concerns the use of an assistive technology platform to facilitate a high level of interaction between an AMD user with one or more caregiver, family member, or healthcare team/provider. The system leverages a low cognitive demand, voice-controlled empathetic relational agent for guidance, navigation, education, social support, social contact, support of daily living activities, safety, support for caregivers, feedback/communication for and between healthcare team/providers, and the like, in the physical rehabilitation and or management of safe and secured assisted mobility. The platform also aids the elderly in overcoming barriers to medication adherence and increases compliance for health and well-being. In one embodiment, the platform or system comprises a combination of at least one of the following components: cellular communication device; computing device; communication network; remote server; cloud server; cloud application software. The cloud server and service are commonly referred to as “on-demand computing”, “software as a service (SaaS)”, “platform computing”, “network-accessible platform”, “cloud services”, “data centers,” and the like. The cloud server is preferably a secured HIPAA-compliant remote server. In an alternative embodiment, the mobility intervention system comprises a combination of at least one: voice-controlled speech user interface; computing device; communication network; remote server; cloud server; cloud application software. These components are configured to function together to enable a user to interact with a resulting relational agent. In addition, an application software, accessible by the user and others, using one or more remote computing devices, provides a social, caregiver, healthcare provider(s) network support system for the user.

The wireless cellular communication device of the present disclosure is a fully functional mobile communication device that is attachable to one or more alternative AMD form factor. The device provides a user-interface that allows a user to access features that include smart and secure location based services, mobile phone module, voice and data, advanced battery system and power management, direct 911 access, and fall detection via an accelerometer sensor. Additional functions may include one or more measurements of linear acceleration, heading, altitude, angular velocity, and angular position. The wearable device may contain one or more microprocessor, microcontroller, micro GSM/GPRS chipset, micro SIM module, read-only memory device (ROM), RAM, memory storage device, I-O devices, buttons, display, user interface, rechargeable battery, microphone, speaker, audio CODEC, power gauge monitor, wireless battery charger, wireless transceiver, antenna, accelerometer, vibrating motor, LEDs, preferably in combination, to function fully as a wearable mobile cellular phone.

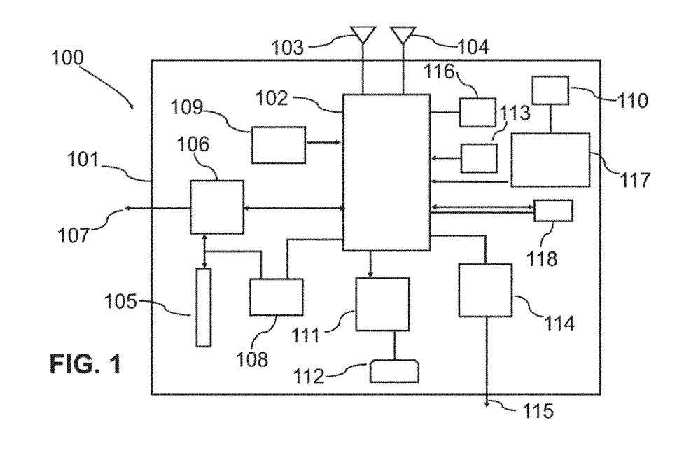

Referring to FIG. 1, schematic diagram 100 illustrates the components that may be incorporated, but are not limited to, within the cellular communication device according to aspects of the present disclosure. Device 101 may contain a radio module 102 that is capable of functioning as a stand-alone wireless cellular communication (e.g., sans Bluetooth) apparatus via connection to cellular antenna 103 and GPS antenna 104. The said communication antennas may be incorporated within device 101 or located external to device 101. The device is powered by a rechargeable battery 105 which can be re-energized by charger 106 in conjunction with an external docking station accessible through direct contact or wireless connection 107. A fuel/power gauge 108 allows the device to monitor energy consumption and life of battery 105. A switched-mode power supply unit 109 and low dropout regulator (LDO) 110 may be incorporated to convert electrical power efficiently, eliminate switching noise, and provide simplicity in design. Device 101 contains also an audio codec 111 functioning together with an audio input-output (I/O) 112. Additional I/O devices may include a connection to one or more I/O buttons 113, output light-emitting diodes (LEDs) via LED driver 114 through connection 115, and vibrational motor 116. In addition, device 101 may contain an accelerometer unit 117 as well as a subscriber identification module (SIM) 118. Device 101 and one or more of its internal components, connected to external components, preferably when attached to one or more AMDs, operate together to augment the functions and capabilities of AMDs. It is understood that the communication device, hardware, and internal components can be integrated within a variety of form factors. For illustration purposes, the physical form factors may include, but are not limited to, an apparatus according to an aspect of the present disclosure, a self-contained, compact box, a miniature device, a device resembling a portable mobile unit, a cellular phone, a mobile phone, a tablet, or the like.

The said wireless communication may include a cellular communication that uses at least one of long-term evolution (LTE), LTE advanced (LTE-A), code division multiple access (CDMA), wideband CDMA (WCDMA), universal mobile telecommunication system (UMTS), wireless broadband (WiBro), and global system for mobile communication (GSM). The wireless communication may include at least one of wireless fidelity (WIFI), Bluetooth™, Bluetooth low energy (BLE), ZigBee™, near field communication (NFC), magnetic secure transmission, radio frequency (RF), or body area network (BAN). The wireless communication may include a global positioning system (GPS), global navigation satellite system (GNSS), Beidou navigation satellite system (Beidou), Galileo, and the European global satellite-based navigation system. Herein, “GPS” may be interchangeably referred to as “GNSS.” Additional bands and equivalent terminologies include Third Generation (3G), Fourth Generation (4G), Fifth Generation (5G), future generations, and the like.

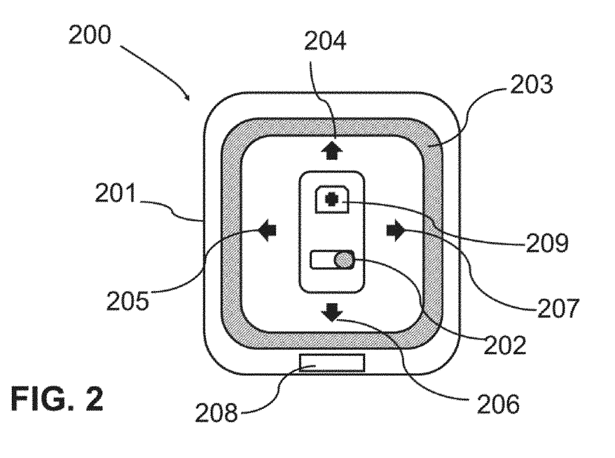

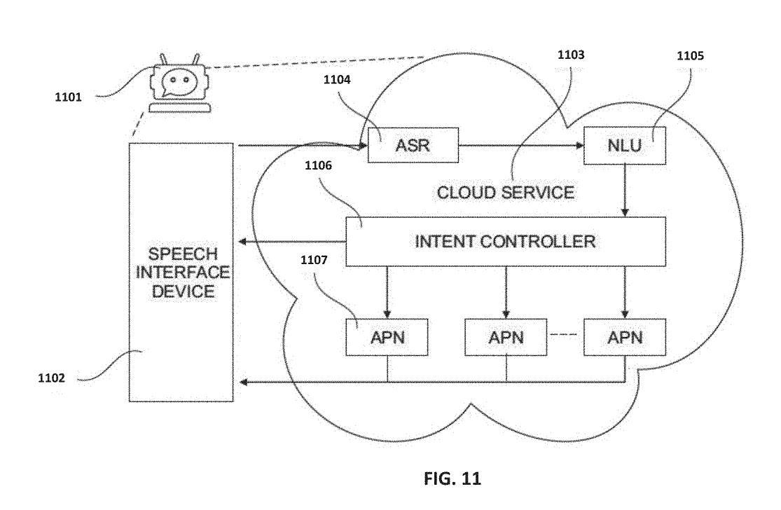

In a preferred embodiment, said device 101 operates in conjunction with an integrated external-facing simple user interface, comprising one or more input buttons and LEDs for user interaction, that is user-friendly, natural, and low cognitive demand. FIG. 2 is an illustration 200 of a preferred simple user interface 201 of said device 101 of FIG. 1. The simple user interface 201 may incorporate an easily recognizable on-off button 202 and a LED ring 203 that is on/lit (i.e., emitting light) when the device is active and allows a user with sensory deficit (e.g., seeing difficulty) to know that the device is on or in active mode. The user interface can also incorporate a directional forward LED 204, a backward LED 205, a left LED 206, and a right LED 207. These LEDs may emit light intermittently or continuously in conjunction with audio outputs provided by device 101 of FIG. 1, functioning as visual aids for guidance or navigation for the AMD user. The outputs may be directions provided by the interactions between the relational agent 1101 (shown in FIG. 11) through a voice-controlled speech user interface via I/O 208. In an implementation of the present disclosure, the relational agent, through the capabilities of the said communication device 101, provides audio-visual guidance and directional navigation instructions via GPS data afforded by the functions of said communication device. In a preferred embodiment, the interface contains a button 209 for direct access to 911 emergency services in the event of an AMD user fall. It is understood that alternative configurations of the user interface may comprise additional I/O components, features, and options, as to expand the functions and capabilities provided to the user of AMDs.

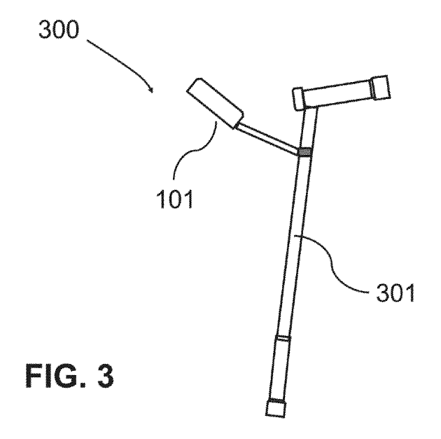

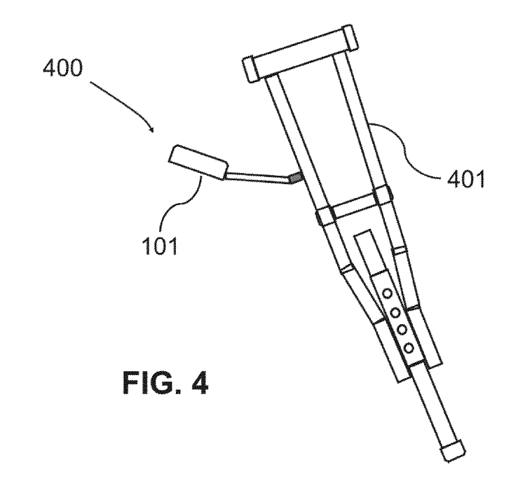

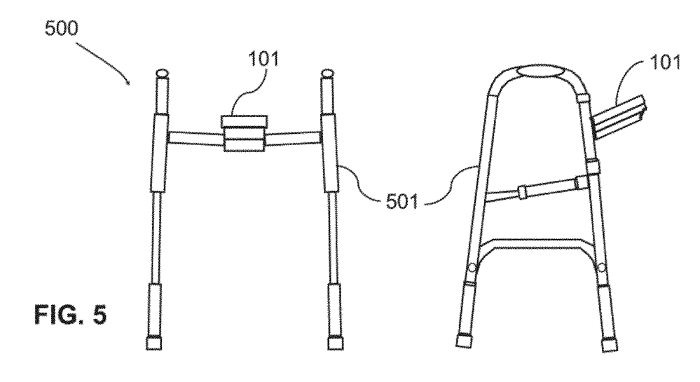

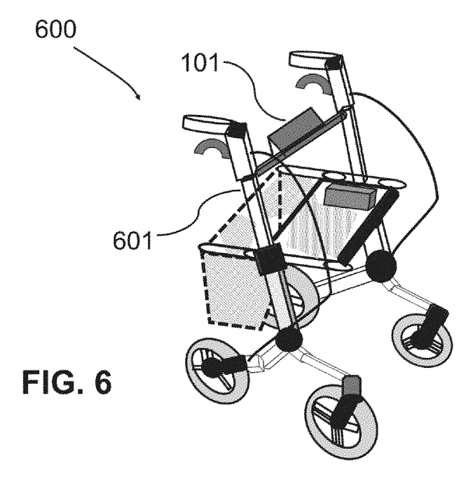

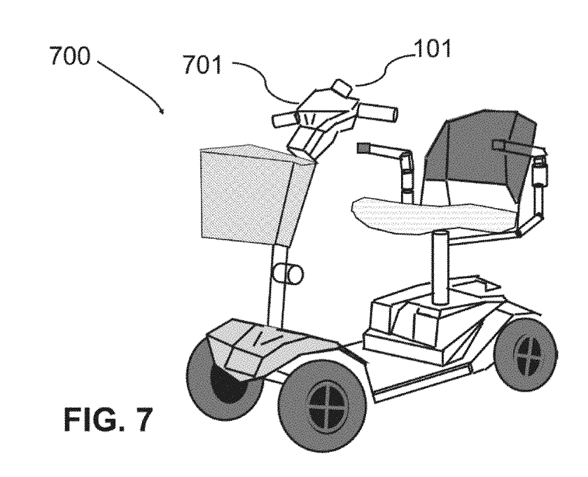

The device 101 of FIG. 1 comprising, for example, user interface 201 of FIG. 2, may be attachable to one or more AMDs to augment their functions and capabilities. Referring to FIG. 3, illustration 300 depicts a conventional cane 301 with device 101 of FIG. 1 attached to its frame. Device 101 operates as a self-contained, compact, battery operated, stand-alone wireless communication unit, providing augmented functions and capabilities to a user of the conventional cane 301. Referring to FIG. 4, illustration 400 depicts device 101 attached to a conventional crutch 401, converting said crutch to an AMD with augmented functions and capabilities. Referring to FIG. 5, illustration 500 depicts a front view and side with device 101 attached to a conventional walker 501, converting said walker to an AMD with augmented functions and capabilities. Referring to FIG. 6, illustration 600 depicts device 101 attached to a conventional rollator 601, converting said rollator to an AMD with augmented functions and capabilities. Referring to FIG. 7, illustration 700 depicts device 101 attached to a conventional scooter 701, converting said scooter to an AMD with augmented functions and capabilities. It is understood that one or more alternative attachment means (e.g., clamp, screws, etc.) may be incorporated to enable secured coupling of device 101 to each said AMD, each attachment means appropriately chosen for each AMD physical form factor.

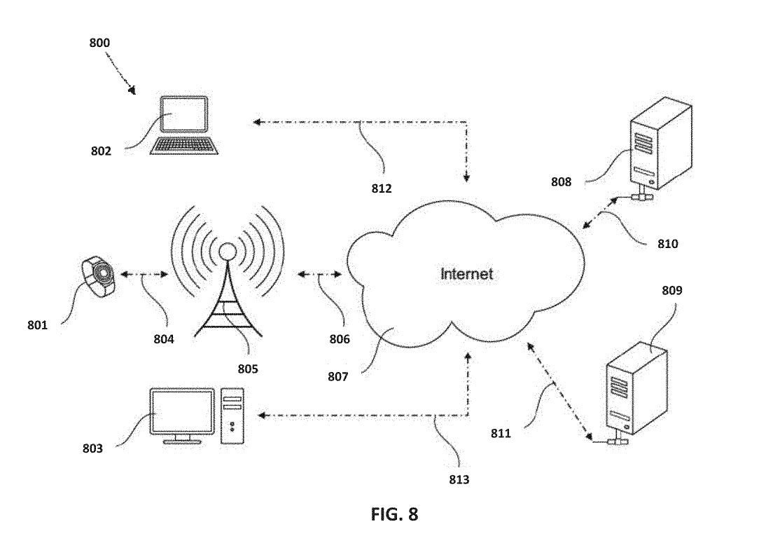

The integrated assistive technology system according to aspects of the present disclosure utilizes an application software platform to create an ecosystem for communication and social networking between one or more AMD user, family, caregivers, and or healthcare providers. Referring to FIG. 8, illustration 800 describes the elements of said ecosystem. One or more user can access the system using a portable computing device 802 or stationary computing device 803. Computing device 802 may be a laptop used by a family member or caregiver. Stationary computing device 803 may reside at the facility of a healthcare provider (e.g., physician's office, physical therapy clinic). Device 801, corresponding to device 101 of FIG. 1, communicates with the system via communication means 804 to one or more cellular communication network 805 which can connect device 801 via communication means 806 to the Internet 807. Device 801, 802, and 803 can access one or more remote servers 808, 809 via the Internet 807 through communication means 810 and 811 depending on the server. Device 802 and 803 can access one or more servers through communication means 812 and 813. The application software platform can be stored in one or more said servers 808, 809. The software environment allows for, but is not limited to, daily tracking of user location, monitoring AMD user medication adherence, monitoring AMD user medication persistence, storing and tracking health data (i.e. blood pressure, glucose, cholesterol, etc.), monitoring/predicting falls, gait patterns, sending-receiving text messages, push notification, sending-receiving voice messages, sending-receiving videos, streaming instructional videos, scheduling doctor's appointments, AMD user education information, caregiver education information, feedback to healthcare providers, and the like. The application software can be used to store skills relating to the self-management of mobility rehabilitation. The application software may contain functions for predicting AMD user behaviors, predicting falls, gait analyses, decline in cognitive functions, predicting non-compliance to physical therapy, functions for suggesting corrective actions, functions to improve cognition, games to improve cognition, games to improve physical coordination, functions to perform or teach interventions for rehabilitation or safe and secured management of mobility. The application software may interact with an electronic health or medical record system (e.g., EMR).

In a preferred embodiment, the said device 801, corresponding to device 101 of FIG. 1, enables communication with one or more remote servers, for example server 808, capable of providing cloud-based control service, to perform natural language or speech-based interaction with the user. The said device 801 detection audio inputs/listens and interacts with a user to determine a user intent based on natural language understanding of the user's speech. The said device 801 is configured to capture user utterances and provide them to the control service located on server 808. The control service performs speech recognition-response and natural language understanding-processing on the utterances to determine intents expressed by the utterances. In response to an identified intent, the controlled service causes a corresponding action to be performed. An action may be performed at the control service or by instructing the said device 801 to perform a function. The combination of the said device 801 and control service located on remote server 808 serve as a relational agent. The relational agent provides conversational interactions, utilizing automated voice recognition-response, natural language processing, predictive algorithms, and the like, to: perform functions, interact with the user, fulfill user requests, educate user, monitor user compliance, determine user health status, user well-being, suggest corrective user actions-behaviors, and the like. The relational agent may fulfill specific requests including calling a family member, a healthcare provider, or arranging a ride (e.g., Uber, Circulation) for the user. In an emergency, for example, a fall event, the relational agent may contact an emergency service. Ultimately the said device 801 enables the user to access and interact with the said relational agent to provide AMD user education, support, social contact support, support of daily activities, user safety, record diary entry/contents, support for caregivers, feedback/communication for healthcare team/providers, and the like, in the self-management of physical rehabilitation or management of safe and secured mobility. The information generated from the interaction of the user and others can be captured and stored in a remote server, for example remote server 809. This information may be incorporated into the application software making it accessible to multi-users (e.g. healthcare team, caregiver, etc.) of the ecosystem herein disclosed.

In an alternative embodiment, the function of the relational agent can be accessed through a mobile app and implemented through a system illustrated in FIG. 8. Such mobile app provide access to a remote, for example remote server 808 of FIG. 8, capable of providing cloud-based control service, to perform natural language or speech-based interaction with the user. The mobile app contained in a mobile cellular unit (e.g., mobile phone) monitors and captures voice commands and or utterances and transmit them through the said communication means to the control service located on server 808. The control service performs speech recognition-response and natural language understanding-processing on the utterances to determine intents expressed by the utterances. In response to an identified intent, the control service causes a corresponding action to be performed. An action may be performed at the control service or by responding to the user through the mobile app. The control service located on remote server 808 serve as a relational agent. The relational agent provides conversational interactions, utilizing automated voice recognition-response, natural language processing, predictive algorithms, and the like, to perform functions, interact with the user, fulfill user requests, educate, monitor compliance, determine health status, well-being, suggest corrective actions-behaviors, and the like. Ultimately the said device enables the user to access and interact with the said relational agent for the self-management of physical rehabilitation or safe and secure management of mobility. The information generated from the interaction of the user and the relational agent can be captured and stored in a remote server, for example remote server 809. This information may be incorporated into the application software as described in FIG. 8, making it accessible to multi-users of the ecosystem herein disclosed.

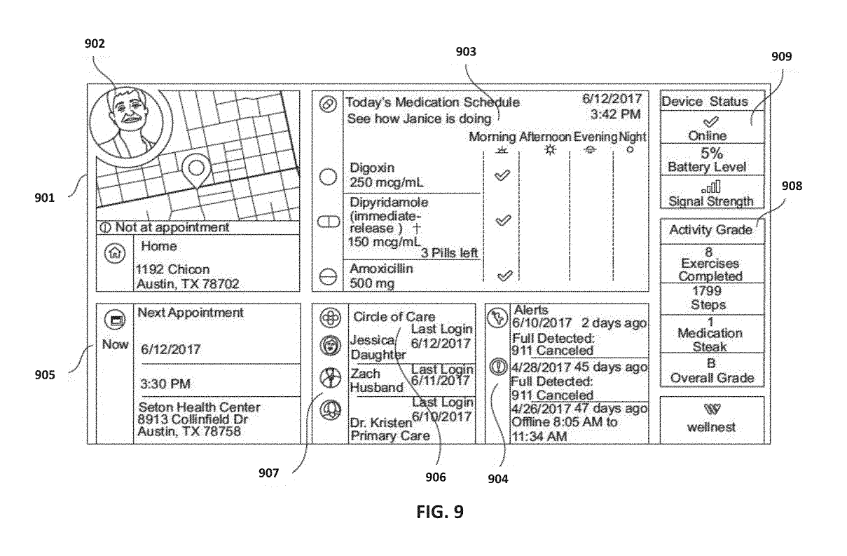

FIG. 9 is a screen-shot 901 that illustrates the type of information that users can generate using the application software platform. Screen-shot 901 provides an example of the information arranged in a specific manner and by no means limits the potential alternative or additional information that can be made available and displayed by the application software. In this example, a picture of an AMD user 902 is presented at the upper left corner. The application may display the current location of the AMD user 902, providing real-time location of the user. A Medication Schedule 903 is available for review and contains a list of medications, dosage, and time when taken. This may be useful for caregivers and healthcare providers in the monitoring of user compliance. An Alerts 904 is also visible that documents fall detection events and 911 emergency connections for the AMD user 902. The AMD user can review the Next Appointment 905 information, for example for returning to a physical therapy session. A Circle of Care 906 has pictures of the people 907 (i.e. family members) interacting with the AMD user 902 in the said ecosystem and log-in information. There is also an Activity Grade 908 that allows users to monitor for example the physical activities of the AMD user 902 using the step count function of the communication device 901. Device Status 909 provides information on the status of said device.

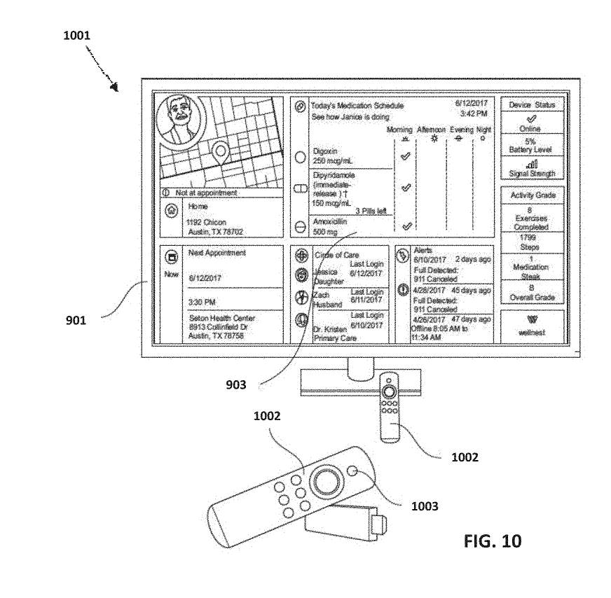

FIG. 10 illustrates the assistive technology platform (system) incorporating a multimedia device 1001 for an AMD user or the like to interact with one or more remote healthcare team/provider, caregiver, or family member through a relational agent. One or more user can access the system using a remote-controlled device 1002 containing a voice-controlled speech user interface 1003. The multimedia device 1001 is configured to enable a user to access application software platform depicted by screen-shot 901. The multimedia device 1001 may be configured with hardware and software that enable streaming videos to be displayed. Exemplary products include FireTV, Fire HD8 Tablet, Echo Show; products available from Amazon.com (Seattle, Wash.), Nucleus (Nucleuslife.com), Triby (Invoxia.com), TCL Xcess, and the like. Streaming videos may include educational contents or materials to improve AMD user behavior, cognition, knowledge, attitudes, and practices to improve adherence and physical therapy outcomes, or general health outcomes. Preferable materials include contents and tools to increase user knowledge and understanding of gait and or mobility; nutritional influences on mobility; physical therapy goals; or improvement of user's planning ability and capability to navigate in familiar and or unfamiliar environments, including but not limited to, yard, neighborhood, cities, metropolitans, or the like.

FIG. 11 illustrates a figurative relational agent 1101 comprising the voice-controlled speech user interface device 1102, corresponding to device 101 of FIG. 1, and a cloud-based control service 1103. A representative cloud-based control service can be implemented through a SaaS model or the like. Model services include, but are not limited to, Amazon Web Services, Amazon Lex, Amazon Lambda, and the like, available through Amazon (Seattle, Wash.). Such a service provides access to one or more remote servers containing hardware and software to operate in conjunction with said voice-controlled speech interface device, app, or the like. Without being bound to a specific configuration, said control service may provide speech services implementing an automated speech recognition (ASR) function 1104, a natural language understanding (NLU) function 1105, an intent router/controller 1106, and one or more applications 1107 providing commands back to the voice-controlled speech interface device, app, or the like. The ASR function can recognize human speech in an audio signal transmitted by the voice-controlled speech interface device received from a built-in microphone. The NLU function can determine a user intent based on user speech that is recognized by the ASR components. The speech services may also include speech generation functionality that synthesizes speech audio. The control service may also provide a dialog management component configured to coordinate speech dialogs or interactions with the user in conjunction with the speech services. Speech dialogs may be used to determine the user intents using speech prompts. One or more applications can serve as a command interpreter that determines functions or commands corresponding to intents expressed by user speech. In certain instances, commands may correspond to functions that are to be performed by the voice-controlled speech user interface device, and the command interpreter may in those cases provide device commands or instructions to the voice-controlled speech user interface device for implementing such functions. The command interpreter can implement “built-in” capabilities that are used in conjunction with the voice-controlled speech user interface device. The control service may be configured to use a library of installable applications including one or more software applications or skill applications according to aspects of the present disclosure. The control service may interact with other network-based services (i.e. Amazon Lambda) to obtain information, access additional database, applications, or services on behalf of the user. A dialog management component is configured to coordinate dialogs or interactions with the user based on speech as recognized by the ASR component and or understood by the NLU component. The control service may also have a text-to-speech component responsive to the dialog management component to generate speech for playback on the voice-controlled speech user interface device. These components may function based on models or rules, which may include acoustic models and specify grammar, lexicons, phrases, responses, and the like created through various training techniques. The dialog management component may utilize dialog models that specify logic for conducting dialogs with users. A dialog comprises an alternating sequence of natural language statements or utterances by the user and system generated speech or textual responses. The dialog models embody logic for creating responses based on received user statements to prompt the user for more detailed information of the intents or to obtain other information from the user. An application selection component or intent router identifies, selects, and/or invokes installed device applications and/or installed server applications in response to user intents identified by the NLU component. In response to a determined user intent, the intent router can identify one of the installed applications capable of servicing the user intent. The application can be called or invoked to satisfy the user intent or to conduct further dialog with the user to further refine the user intent. Each of the installed applications may have an intent specification that defines the serviceable intent. The control service uses the intent specifications to detect user utterances, expressions, or intents that correspond to the applications. An application intent specification may include NLU models for use by the natural language understanding component. In addition, one or installed applications may contain specified dialog models that create and coordinate speech interactions with the user. The dialog models may be used by the dialog management component in conjunction with the dialog models to create and coordinate dialogs with the user and to determine user intent either before or during operation of the installed applications. The NLU component and the dialog management component may be configured to use the intent specifications of the applications either to conduct dialogs, to identify expressed intents of users, identify and use the intent specifications of installed applications, in conjunction with the NLU models and dialog modes, to determine when a user has expressed an intent that can be serviced by the application, and to conduct one or more dialogs with the user. As an example, in response to a user utterance, the control service may refer to the intent specifications of multiple applications, including both device applications and server applications, to identify a “Wellwalker” intent. The service may then invoke the corresponding application. Upon invocation, the application may receive an indication of the determined intent and may conduct or coordinate further dialogs with the user to elicit further intent details. Upon determining sufficient details regarding the user intent, the application may perform its designed functionality in fulfillment of the intent. The voice-controlled speech interface device in combination with one or more functions 1104, 1105, 1106 and applications 1107 provided by the cloud service represents the relational agent 1101.

In a preferred embodiment, skills are developed for the relational agent 1101 of FIG. 11 and stored as accessible applications within the cloud service 1103. The skills contain information that enables the relational agent to respond to intents by performing an action in response to a natural language user input, information of utterances, spoken phrases that a user can use to invoke and intent, slots or input data required to fulfill an intent, and fulfillment mechanisms for the intent. These application skills may also reside in an alternative remote service, remote database, the Internet, or the like, and yet accessible to the cloud service 1103. These skills may include but are not limited to intents for general topics, weather, news, music, pollen counts, UV conditions, user/patient engagement skills, nutrition, instructions for taking medication, prescription instructions, medication adherence, persistence, coping skills, cognitive skills, physical rehabilitation skills, game skills to improve cognitive functions, game skills to improve physical coordination, navigation skills, behavioral skills, risk stratification skills, daily activity living skills, and the like. The skills enable the relational agent 1101 to respond to intents and fulfill them through the voice-controlled speech interface device. These skills may be developed using application tools from vendors (i.e. Amazon Web Services, Alexa Skill Kits) providing cloud control services. The AMD user preferably interacts with relational agent 1101 using skills that enable a voluntary, active, and collaborative effort between an AMD user with caregivers, family members, or health care team/providers.

Exemplary skills accessible to a patient may be one or more physical rehabilitation or mobility management interventions including self-efficacy skills, self-management skills, medication adherence skills, cognitive enhancement skills, skills relating to gait, walking skills, physical rehabilitation skills, mobility skills, coping skills, or the like. An implementation of the present disclosure can provide a relational agent with skills to be able to fulfill one or more intents invoked by a patient, for example: symptoms identification (e.g. symptoms of impending fall, etc.) and management skills (e.g., invoke relational agent to call 911). It is a preferred object to utilize the spoken language interface as a natural means of interaction between the users and the system. Users can speak to the assistive technology similarly as they would normally speak to a human. It is understood, but not bound by theory, that verbal communication accompanied by the opportunity to engage in meaningful conversations can reinforce, improve, and motivate behavior for simultaneous self-management of mobility. The relational agent may be used to engage AMD users in activities aimed at stimulating social functioning to leverage social support for improved compliance, persistence, and coping. These skills may create a user-centered environment for user-centered care. Preferred skills include but are not limited to those that examine important psychological or social constructs or utilize informative tools for assessing and improving user mobility. The preferred environment is a collaborative relationship between providers and users, in a shared decision-making and a personal systems approach; both have the potential to improve medication adherence, physical rehabilitation and/or mobility outcomes.

The relational agent and one or more skills may be implemented in the engagement of an AMD user at an ambulatory setting (i.e. home, physician's office, clinic, etc.). During a session, the relational agent using one or more skills may inform the user about, for example, proper walking, or proper usage of an AMD. Skills may include topics of instructions to prevent complications and the effect of cognition, cognition deficit, diet and medication on mobility. The relational agent may inform the patient about the possible problems that might be encountered with operations of an AMD. The platform according to aspects of the present disclosure preferably allows the remote monitoring by a healthcare team/provider (e.g., nurse, clinician, specialist, therapist) on the quality (e.g. mean, standard deviation, frequency, etc.) of the mobility self-management by users/patients and prompts intervention as necessary.

In another aspect of the present disclosure a means is provided to assess knowledge of specific diseases, physical, mental, neural, or cognitive health conditions, monitor medication adherence, and assess the emotional well-being of an AMD user using a standard set of validated questionnaires and or patient-reported outcomes (PROs) instruments. The responses-answers provided or obtained from these questionnaires and instruments enable the assessment of a user's physical functioning, psychological functioning, and overall health-related QoL. One or more questionnaires and answer-responses may be on the topic of self-efficacy or confidence in the ability to perform walks, manage mobility or related complications. This may be implemented using clinically validated questionnaires conducted by the relational agent. Upon a user intent, the relational agent can execute an algorithm or a pathway consisting of a series of questions that proceed in a state-machine manner, based upon yes or no responses, or specific response choices provided to the user. For example, a clinically validated structured multi-item, multidimensional, questionnaire scale may be used to assess knowledge of physical therapy, health conditions, or symptoms, self-efficacy, or the like. The scale is preferably numerical, qualitative or quantitative, and allows for concurrent and predictive validity, with high internal consistency (i.e., high Cronbach's alpha), high sensitivity and specificity. Questions are asked by the relational agent and responses, which may be in the form of yes/no answers from patients or caregivers, are recorded and processed by one or more skills. Responses may be assigned a numerical value, for example yes=1 and no=0. A high sum of yes in this case provides a measure of non-adherence. One of ordinary skill in the art can appreciate the novelty and usefulness of using the relational agent according to aspects of the present disclosure; a voice-controlled speech recognition and natural language processing combined with the utility of validated clinical questionnaire scales or PROs instruments. The questionnaire scales are constructed and implemented using skills developed through for example using the Alexa Skills Kit and or Amazon Lex. The combination of these modalities may be more conducive to eliciting information, providing feedback, and actively engaging AMD users and caregivers for the self-management of mobility. When a user reports symptoms, the relational agent probes them to provide specific information about each symptom with multiple choice questions, and based on user responses, provides personalized management recommendations.

Clinically validated scales and PROs instruments may be constructed to measure, assess, or monitor the following, without being limited to: physical well-being, social well-being, emotional well-being, functional well-being, pain, fatigue, nausea, sleep disturbance, distress, shortness of breath, loss of memory, loss of appetite, drowsiness, dry mouth, anxiety, sadness, emesis, numbness, bruising, disease specific-related symptoms, or the like; rated on the basis of their presence and severity. PROs instruments may also be constructed to measure, assess, or monitor medication, medication administration, medication interactions, activity, diet, side effects, informing healthcare team/providers, informing therapists, and QoL. It is understood that any clinically validated PROs instruments, modified or unmodified, for the management of cognition and mobility may be implemented according to aspects of the present disclosure. All said questionnaires, PRO instruments, scales and the like can be constructed and implemented using the Alexa Skills Kit and or Amazon Lex system, or the like. User responses provide objective data about different aspects of education and practice that are taught and retained by the user education. Thus, these instruments, for example, serve as a good quality control measure of user education/counseling/rehabilitation effectiveness. Frequently missed questions may indicate potential areas for improvement in user education, including reinforcement of treatment guidelines as well as recommendation to contact healthcare providers or therapists for questions. In addition, the relational agent may assess the need for re-education or suggest areas for improvements to keep patients in compliance with physical therapy.

The said scales may be modifiable with a variable number of items and may contain sub-scales with either yes/no answers, or response options, response options assigned to number values, Likert-response options, or Visual Analog Scale (VAS) responses. VAS responses may be displayed via mobile app in the form of text messages employing emojis, digital images, icons, and the like.

The results from one or more questionnaire, scales, and PROs instruments may be obtained and/or combined to monitor and provide support for AMD user education, social contact, daily activities, user safety, support for caregivers, and feedback communication for healthcare providers in the self-management of physical rehabilitation/therapy or safe and secured management of mobility. Questionnaire, scales, and PROs instruments may be directed to either caregivers or AMD users. User responses on the questionnaires are sent to the application software platform. The answers provided to the relational agent serve as input to one or more indices, predictive algorithms, statistical analyses, or the like, to calculate a risk stratification profile and trends. Such a profile can provide an assessment for the need of any intervention (i.e. corrective action) required by either the user, healthcare team/providers, caregivers, or family members. Trends in these symptoms can be recorded and displayed in a graphical format within the application software.

In summary, the device and the assistive technology platform (system) disclosed herein serve to augment the functions and capabilities of AMDs and provide a social, caregiver, healthcare provider(s) network support system for the user. The system incorporates a voice-controlled empathetic relational agent as a simple, user-friendly, natural, and low cognitive demand user-interface for guidance, directional navigation, user education, general support, social contact support, support with daily living activities, safety, support for caregivers, feedback for healthcare providers, and the like, in the management of physical rehabilitation, mobility recovery, or safe and secured management of mobility. For AMD users, the system supports their needs regarding, without being limited to, medication adherence, symptoms management, cognitive aid, cognitive stimulation, coping, emotional support, social support, and educational information on for example gait or the use of AMDs. For caregivers, the system supports their needs regarding, without being limited to, information about mobility assistance, AMDs and medication information, advice and emotional support, health conditions, and health information resources. For healthcare team/providers, the system supports their needs regarding, without being limited to, patient behavior, profile, medication adherence, routine adherence, adherence to physical therapy, user/patient health status, and sharing of patient information across multiple healthcare settings (e.g., PCPs, specialists, pharmacists, etc.). The system establishes an ecosystem that is AMD user-centered, comprehensive, coordinated, accessible (24/7), and enables healthcare team/providers to enhance quality improvement, ensuring that AMD users/patients and families make informed decisions about their health. The system has utility to promote secured mobility of elderly people, or people with cognitive and sensory deficits stemming from conditions or diseases (e.g., Dementia, Parkinson's, Alzheimer's, Multiple Sclerosis, Stroke), by improving attention, dual task performance, mood, and executive functions, as well as providing, among others, orientation/navigation guidance.

### Example 1

This example is intended to serve as a demonstration of the possible voice interactions between a relational agent and a user of an enhanced AMD. The relational agent uses a control service (Amazon Lex) available from Amazon.com (Seattle, Wash.). Access to skills requires the use of a device wake word (“Alexa”) as well as an invocation phrase (“Wellwalk”) for skills specifically developed for the said device according to various aspects of the present disclosure. The following highlights one or more contemplated capabilities and uses of implementations of the present disclosure:

| Feature | Sample Phrases |
|---|---|
| Checking | “Alexa, ask Wellwalk if I have any messages” “Alexa, |
| Messages | tell Wellwalk to check my messages” |
| Appointment | “Alexa, ask Wellwalk when my next appointment is.” |
| Schedule | “Alexa, ask Wellwalk about my appointment schedule.” |
| Fire TV | “Alexa, ask Wellwalk what is new on Fire TV about |
| Video | smart walkers” |
| Content | “Alexa, ask Wellwalk if there is anything new on Fire TV |
| | about how to use my rollator” |
| Navigation | “Alexa, ask Wellwalk where is the nearest coffee shop |
| Help | (conversation will continue) |
| | “Alexa, ask Wellwalk how far is the nearest wheel chair |
| | ramp (conversation will continue) |
| | “Alexa, ask Wellwalk to guide me to the park |
| | (conversation will continue) |
| | Alexa: “Walk forward 100 yards and turn left . . . ” |
| | (conversation will continue) |
| Emergency | “Alexa, tell Wellwalk to call 911. I have fallen |
| Assistance | and can't get up” |
| | “Alexa, ask Wellwalk to call an ambulance” |
| Contact | “Alexa, tell Wellwalk to call Hannah” |
| Family | |
| Medication | “Alexa, tell Wellwalk I'd like to ask how I am |
| Adherence | doing with my medication” |
| Measure | Alexa: “I would like to ask you several guestions. |
| | Would you like to proceed?” |
| | Alexa: “Do you remember what you should do when |
| | realizing that you have forgotten to take your medicine?” |

Many different embodiments have been disclosed regarding the above descriptions and the drawings. It will be understood that it would be unduly repetitious to literally describe and illustrate every combination and sub-combination of these embodiments. Accordingly, the present specification, including the drawings, shall be construed to constitute a complete written description of all combinations and sub-combinations of the embodiments described herein, and of the manner and process of making and using them, and shall support claims to any such combination or sub-combination. In the drawings and specification, there have been disclosed various embodiments and, although specific terms are employed, they are used in a generic and descriptive sense only and not for purposes of limitation. Therefore, it will be apparent to those skilled in the art that various modifications and variations can be made to the disclosed embodiments without departing from the scope or spirit of the present disclosure. All such modifications and variations are intended to be included herein within the scope of the present disclosure, and all possible claims to individual aspects or combinations of elements or steps are intended to be supported by the present disclosure.

What is claimed is:

1. An assistive mobility system comprising:
a portable user interface device operably engaged with a communications network, the portable user interface device configured to be selectively coupled to a surface of an assistive mobility device, the portable user interface device comprising at least one processor operably engaged with at least one non-transitory computer readable medium, a user interface comprising one or more input/output means operably engaged with the at least one processor, at least one accelerometer engaged with the at least one processor, at least one microphone and at least one speaker operably engaged with the at least one processor, the at least one non-transitory computer readable medium having instructions stored thereon to cause the processor to process one or more user inputs, and perform one or more assistive mobility actions in response to the one or more user inputs; and,
a remote application server being communicably engaged with the portable user interface device via the communications network to receive a data transmission comprising the one or more user inputs, the remote application server executing a control service comprising an automated speech recognition function, a natural-language processing function, and an application software, the application software executing one or more routines in response to the data transmission, the one or more routines comprising instructions for delivering one or more assistive mobility prompts to the portable user interface device.

2. The system of claim 1 wherein the one or more assistive mobility actions comprise one or more audio-visual outputs comprising a directional guidance or physical therapy prompt.

3. The system of claim 1 wherein the user interface further comprises a plurality of directional indicators, the plurality of directional indicators being operably engaged with the processor to provide one or more directional guidance or navigational outputs.

4. The system of claim 1 further comprising a personal computing device communicably engaged with the remote application server via the communications network to access an instance of the application software via a web or mobile browser, the instance of the application software being configured to assemble a graphical user interface to display a plurality of user activity data or health data.

5. The system of claim 1 wherein the remote application server is communicably engaged with an electronic health or medical record server.

6. The system of claim 1 wherein the one or more assistive mobility prompts comprise one or more physical rehabilitation or mobility management interventions.

7. The system of claim 1 wherein the one or more routines further comprise instructions for evaluating a user gait pattern and predicting a likelihood of a user fall.

8. The system of claim 1 wherein the portable user interface device further comprises a vibrating motor operably engaged with the at least one processor, the vibrating motor being configured to provide a haptic feedback to a user in response to one or more physical rehabilitation or mobility management interventions.

9. An assistive mobility system comprising:
an assistive mobility device;
a portable user interface device operably engaged with a communications network, the portable user interface device being selectively coupled to a surface of the assistive mobility device, the portable user interface device comprising at least one processor operably engaged with at least one non-transitory computer readable medium, a user interface comprising one or more input/output means operably engaged with the at least one processor, at least one accelerometer engaged with the at least one processor, at least one microphone and at least one speaker operably engaged with the at least one processor, the at least one non-transitory computer readable medium having instructions stored thereon to cause the processor to monitor a plurality of user activity data and execute one or more physical rehabilitation or mobility management interventions in response to the plurality of user activity data; and,
a remote application server being communicably engaged with the portable user interface device via the communications network to receive the plurality of user activity data, the remote application server executing a control service comprising an automated speech recognition function, a natural-language processing function, and an application software, the application software executing one or more routines in response to the plurality of user activity data, the one or more routines comprising instructions for evaluating user compliance with one or more physical rehabilitation or mobility management parameters.

10. The system of claim 9 wherein the assistive mobility device is selected from the group consisting of canes, walkers, rollators, wheelchairs, and scooters.

11. The system of claim 9 further comprising a personal computing device communicably engaged with the remote application server via the communications network to access an instance of the application software via a web or mobile browser, the instance of the application software being configured to assemble a graphical user interface to display the plurality of user activity data.

12. The system of claim 11 wherein the one or more routines further comprise instructions for communicating a notification to the personal computing device in response to an instance of non-compliance with the one or more physical rehabilitation or mobility management parameters.

13. The system of claim 11 wherein the one or more routines further comprise instructions for evaluating a user gait pattern and predicting a likelihood of a user fall.

14. The system of claim 13 wherein the one or more routines further comprise instructions for communicating a notification to the personal computing device in response to an instance of a prediction of a user fall.

15. The system of claim 9 wherein the remote application server is communicably engaged with an electronic health or medical record server.

16. An assistive mobility system comprising:
an assistive mobility device comprising an assistive mobility assembly and an electronics assembly being operably engaged with a communications network, the electronics assembly comprising at least one processor operably engaged with at least one non-transitory computer readable medium, a user interface comprising one or more input/output means operably engaged with the at least one processor, at least one accelerometer engaged with the at least one processor, at least one microphone and at least one speaker operably engaged with the at least one processor, the at least one non-transitory computer readable medium having instructions stored thereon to cause the processor to monitor a plurality of user activity data and execute one or more physical rehabilitation or mobility management interventions in response to the plurality of user activity data; and,
a remote application server being communicably engaged with the assistive mobility device via the communications network to receive the plurality of user activity data, the remote application server executing a control service comprising an automated speech recognition function, a natural-language processing function, and an application software, the application software executing one or more routines in response to the plurality of user activity data, the one or more routines comprising instructions for evaluating user compliance with one or more physical rehabilitation or mobility management parameters.

17. The system of claim 16 wherein the user interface further comprises a plurality of directional indicators, the plurality of directional indicators being operably engaged with the processor to provide one or more directional guidance or navigational outputs.

18. The system of claim 16 further comprising a personal computing device communicably engaged with the remote application server via the communications network to access an instance of the application software via a web or mobile browser, the instance of the application software being configured to assemble a graphical user interface to display the plurality of user activity data.

19. The system of claim 18 wherein the one or more routines further comprise instructions for evaluating a user gait pattern and predicting a likelihood of a user fall.

20. The system of claim 19 wherein the one or more routines further comprise instructions for communicating a notification to the personal computing device in response to an instance of a prediction of a user fall.
